(19) United States
(12) Patent Application Publication
(10) Pub. No.: US 2003/0124558 A1
(43) Pub. Date: Jul. 3, 2003

(54) Cadherin-11 as an indicator of viable pregnancy

(76) Inventors: Colin D. MacCalman, Vancouver (CA); Mary D. Stephenson, Vancouver (CA)

Correspondence Address: MARSHALL, GERSTEIN & BORUN, 6300 SEARS TOWER, 233 SOUTH WACKER, CHICAGO, IL, 60606-6357, US

(21) Appl. No.: 10/163,818

(22) Filed: Jun. 5, 2002

Related U.S. Application Data
(63) Continuation of application No. 09/403,441, filed on Feb. 15, 2000, now abandoned.
Application No. 09/403,441 is a 371 of international application No. PCT/CA98/00397, filed on Apr. 24, 1998.

(30) Foreign Application Priority Data
Apr. 25, 1997 (CA) 2,203,718

(51) Int. Cl. C12Q 1/68; G01N 33/53

(52) U.S. Cl. 435/6; 436/510

(57) ABSTRACT

Novel diagnostic/monitoring methods are provided using cadherin-11 expression by endometrial tissue as indicator of ability to establish or maintain a viable pregnancy.

### FIELD OF THE INVENTION

This invention relates to production of cadherin-11 in human endometrial tissue as an indicator of ability to establish or maintain a viable pregnancy.

### BACKGROUND OF THE INVENTION

The molecular defects responsible for common reproductive health problems such as infertility and habitual or recurrent spontaneous abortion are not fully known. However, it is believed that implantation failure may contribute to a substantial proportion of cases. In addition, despite increasing experience with assisted reproduction technologies, a low proportion of women undergoing in vitro fertilization and embryo transfer establishes a viable pregnancy. A limiting factor in this setting may be the ability of the (pre-embryonic) blastocyst to attach and/or invade the uterine endometrium, process called implantation.

Recurrent spontaneous abortion (RSA), defined as two or more consecutive pregnancy losses under 20 weeks of gestation, is a prevalent health problem affecting up to 5% of couples trying to establish a family. Genetic (translocations of either partner), endocrine (thyroid disease, hyperprolactinemia or luteal phase deficiency) anatomical (septate uterus, intrauterine adhesions or a submucous fibroid) and autoimmune factors (the Antiphospholipid Antibody (APA) or the Undifferentiated Connective Tissue (UCT) Syndromes) are known risk factors for RSA. The majority (80%) of endocrine-associated RSA is the result of a luteal phase deficiency (LPD), defined as two “out-of-phase” endometrial biopsies, each consisting of ≧3 days of maturation delay according to endometrial morphology and the onset of the next menses.

To establish a successful pregnancy, the trophoblast cells of the pre-embryonic blastocyst must interact with the uterine endometrium during a defined period of the menstrual cycle (called the window of implantation). outside of this receptive period, the endometrium discourages implantation. Aberrant or “out of phase” development of the endometrium during the menstrual cycle has been associated with implantation failure, one of the factors believed to be underlying RSA and infertility. The adhesive mechanisms involved in establishing a uterine environment which promotes trophoblast-endometrial cell interactions have been poorly characterized.

The first step in human implantation involves the attachment of the trophoblast cells of the pre-embryonic blastocyst to the surface epithelium of the uterine endometrium. Afterwards, the trophoblast cells proliferate and invade into the underlying endometrial stroma. The trophoblast cells differentiate into chorionic villi which are composed of two layers: the inner cell layer, which comprises mitotically active cytotrophoblasts, and the outer syncytial trophoblast, which is a terminally differentiated multi-nucleated cell formed by the fusion of post-mitotic cytotrophoblasts. As pregnancy proceeds, the cytotrophoblasts proliterate and form columns which extend through the syncytial trophoblast layer into the maternal decidua. These extravillous cytotrophoblast columns are believed to anchor the placenta to the decidua. Cytotrophoblasts dissociate from the extravillous columns and invade deeply into the maternal vasculature and decidua. These invasive cytotrophoblasts subsequently undergo differentiation and fusion to form placental bed giant cells, large multinucleated cells which lie in intimate contact with the surrounding decidual cells. During invasion of the endometrium, the trophoblast cells must not only interact with one another but with the diverse populations of cell types that constitute the endometrium.

The steroid hormones progesterone (P4) and 17β-estradiol (E2) play a central role in preparing the endometrium for implantation. One of the steps involved in preparing the endometrium involves the differentiation of the stromal cells into decidual cells, which anchor the trophoblast cells and arrest their invasive migration. Morphologically, decidualization is characterized by a change of the stromal cells to a polyhedral cell shape with an increase in cell size. Ultrastructurally, there is extensive development of the organelles involved in protein synthesis (rough endoplasmic reticulum) and secretion (Golgi apparatus), and the appearance of desmosomes and gap junctions between adjacent cells.

The depth of trophoblast invasion is precisely controlled, and errors have extreme consequences to the health of the mother and fetus. For example, shallow invasion is associated with preeclampsia, a disease with significant maternal and fetal morbidity and mortality. In contrast, the absence of decidua allows trophoblasts to invade deeply into the underlying tissue as is the case in placenta accreta or ectopic pregnancy.

The cadherins are a gene superfamily of integral membrane glycoproteins that mediate calcium-dependent cell adhesion in a homophilic manner. The spatiotemporal expression of cadherin subtypes is highly regulated during development. Embryonic cells displaying different classical cadherins (type 1 cadherins) segregate from one another and it is believed that these cadherins provide the molecular basis for the segregation of discrete populations of cells and the subsequent formation of tissues. In-the adult, the cadherins are localized to the membrane domains of the adherens junction and are believed to maintain the differentiated state of the cell.

Type 2 cadherins show low overall amino acid homology with classical cadherins. The type 2 cadherins share common sequence features, such as characteristic amino acid deletions or additions and distinctive amino acid substitutions at various sites, which are not found in classical cadherins. In particular, type 2 cadherins do not contain the cell adhesion recognition (CAR) sequence, HAV, which is conserved among all the classical cadherin subtypes. Cadherin-11 (cad-11), also known as OB-cadherin (OB-cad), is a type 2 cadherin which appears to play a central role in morphogenesis. [See U.S. Pat. No. 5,597,725 issued Jan. 28, 1997, incorporated herein by reference; and, Takeichi, M. (1995) “Morphogenetic Roles of Classical Cadherins”, Curr. Opin. Cell. Biol. 7:619-627.]

It has been determined that cad-11 is expressed in the syncytial trophoblast but not the villous cytotrophoblasts of the human term placenta. Cad-11 expression was also defected in the cytotrophoblasts at the distal end of the extravillous cytotrophoblast column of the first trimester placenta. In the endometrium, cad-11 is spatiotemporally expressed in the glandular epithelium and stroma during the menstrual cycle. Levels of cad-11 in the glandular and surface epithelium remain relatively constant throughout the menstrual cycle. Cad-11 is not present in the stroma during the proliferative phase. Cad-11 is first detected around the spiral arteries of the stroma (the areas of early decidualization) during the late secretory phase. Cad-11 levels increase as the stroma continues to undergo decidualization and maximum levels are observed in the decidua of early pregnancy. [See MacCalman, et al. (1996) “Regulated Expression of Cadherin-11 in KHiman Epithelial Cells: A Role for Cadherin-11 in Trophoblast-Endometrium Interactions?”, Developmental Dynamics 206: 201-211; MacCalman et al., “Novel Cell Adhesion Molecules: Roles in Implantation?” from The Endometrium as a Target for Contraception, Beier et al., eds., Springer-Verlag, Berlin (1996), pages 137-157.]

It has now been found that women presenting with primary infertility and women presenting with habitual abortion show non-existent or significantly reduced levels of cad-11 in the endometrium tissue as compared to fertile women. Women who had presented with habitual abortion but maintained a viable pregnancy following hormonal treatment, exhibited levels of cad-11 in the endometrium comparable to fertile women. Thus, the capacity for cad-11 expression or the production of cad-11 in endometrial tissue, is an indicator of a viable pregnancy. Reduced levels of cad-11 expression/production in endometrial tissue indicates an inability to establish or maintain a pregnancy.

### SUMMARY OF THE INVENTION

This invention provides a method of determining an inability in a woman to establish or maintain a pregnancy, which comprises determination of the presence of a gene encoding normal and functional cad-11 in a tissue sample from the woman.

This invention also provides a method of determining an inability in a subject woman to establish or maintain a pregnancy, which comprises determination of:

(i) cad-11 mRNA, or

(ii) cad-11 protein,

from endometrial cells of the subject woman. Correspondingly provided is a method of determining the ability of a woman to establish or maintain a pregnancy comprising determination of levels of cad-11 mRNA or cad-11 protein from endometrial tissue of a woman. These methods may include comparing the determination from the subject woman to a standard level indicative of ability to establish or maintain a pregnancy in a female. The standard level may be obtained by performing a determination of cad-11 mRNA or protein level in a standard sample of tissue or cells known to express cad-11 (such as placenta, lung, kidney, spleen, testes, ovary, or colon) or endometrial tissue or cells from a known fertile woman. The level of cad-11 mRNA or protein in the standard sample may be determined at the time that the determination of the subject woman is made or the level of the standard may be predetermined and information from the predetermination is compared to the determination made on the subject woman. The standard sample may be artificial. For example, in the case of a cad-11 determination done on serum, cell extracts, etc., the standard may be a prepared solution containing cad-11 mRNA or protein intended to provide a cad-11 determination indicative of a cad-11 level associated with endometrial tissue of a fertile woman.

For proper comparison of a subject woman to a standard level derived from endometrial tissue of a known fertile woman, it is desirable that tissue samples from test women and known fertile women be obtained at approximately the same time during the menstrual cycle, for example in the mid to late secretory phase (days 20-24 of the menstrual cycle). The menstrual cycle begins at the onset of a menstrual bleeding episode and lasts until the onset of the next. In women of reproductive age, the normal menstrual cycle averages 28 days. Thus, day 1 of a cycle would be the first day of menstruation, and day 28 would be the day of the next menstrual bleeding episode. The optimal time of endometrial receptivity to blastocyst or embryo implantation is around days 20-24 of the cycle.

Thus, this aspect of the invention contemplates a method of determining likelihood of establishment or maintenance of a pregnancy comprising determining the level of cadherin-11 production by endometrial cells of a female subject, and comparing said level to a standard level indicative of ability to establish and maintain a pregnancy in a fertile female subject, wherein a reduced level relative to said standard level indicates inability to establish or maintain a pregnancy.

As described in Examples 1 and 2 below, determinations of cad-11 production by endometrium may aid in diagnosing women with habitual abortion or RSA, including women suffering from luteal phase deficiency (LPD), as well as women suffering from infertility.

It is further contemplated that cad-11 protein produced by the endometrium may be cleaved and released into the serum so that the cad-11 protein levels detected in serum can be correlated to cad-l production by the endometrium. Thus, this aspect of the invention may also be practiced by determining cad-11 levels in blood samples, such as serum or plasma, and comparing said level to a standard level in a blood sample from a fertile female subject. As with endometrial tissue samples, blood samples should be taken at approximately the same time during the menstrual cycle.

This invention also provides a kit for performing an immunological determination of cad-11 for determining an inability for a woman to establish or maintain a pregnancy, comprising an antibody or an antibody fragment capable of binding to human cad-11. The kit may include reagents for the detection of the antibody or antibody fragment when .bound to cad-11. The kit may include a container or containers (e.g. commercial packaging) for the components of the kit.

This invention also provides a kit for performing a determination of cad-11 mRNA for determining an inability of a woman to establish or maintain a pregnancy, comprising one or more oligonucleotide primers which hybridizes with cad-11 mRNA. The kit may include reagents for reverse transcription (RT) of the mRNA. The kit may include one or more oligonucleotide primers and reagents for amplification of cDNA from cad-11 mRNA. The kit may include one or more oligonucleotide probes and reagents for the detection of cDNA from cad-11 mRNA. The kit may include a container or containers (e.g. commercial packaging) for the components of the kit.

This invention also provides the use of an antibody or an antibody fragment capable of binding to cad-11 for the detection of cad-11 in endometrial tissue for determining an inability of a woman to establish or maintain a pregnancy, or correspondingly for determining the ability of a woman to establish or maintain a pregnancy.

This invention also provides the use of an oligonucleotide homologous with cad-11 mRNA for the detection of cad-11 mRNA in endometrial tissue for determining an inability of a woman to establish or maintain a pregnancy, or correspondingly for determining the ability of a woman to establish or maintain a pregnancy.

This invention also provides the use of an oligonucleotide homologous with DNA encoding cad-11 in the detection of a cad-11 gene in tissue for determining an inability of a woman to establish or maintain a pregnancy, or correspondingly for determining the ability of a woman to establish or maintain a pregnancy.

This invention also provides the use of progestins to increase cad-11 production in endometrial tissue. Typically, fertility-increasing therapy for patients suffering from endometrial dysfunction involves administering a suitable progestin such as progesterone (eg. P4) pregnenolone or medroxyprogesterone, or an agent that increases progestin levels in a patient (eg. clomiphene or gonadotrophins). Progestins increase cad-11 in endometrial tissue and such treatment may be performed in combination with the methods of this invention whereby cad-11 from endometrial tissue is determined.

As shown in Example 1 herein, the determination of levels of production of cad-11 by endometrium is an indicator of the success of hormonal (progestin) therapy for improving fertility. High expression levels of cad-11 after hormonal fertility therapy were correlated to maintenance of a viable pregnancy. As stated in Example 2 below, cad-11 is a useful marker for luteal phase maturation and endometrial receptivity. Thus, another aspect of the invention contemplates that endometrial cad-11 expression levels are determined and used as a basis for adjusting the dose of progestin-increasing hormones or drugs (e.g., progesterone or clomiphene) administered to women so that the cad-11 production level (a marker for endometrial receptivity) is increased. Although endometrial cell morphology is currently used to titrate hormone dosage, cad-11 expression is indicated herein to be a better predictor of endometrial responsiveness and receptivity. It is contemplated that a physician may monitor the level of endometrial cad-11 expression by a woman undergoing hormonal fertility therapy and adjust dosages so that cad-11 expression approaches the level of cad-11 expression observed in fertile female subjects receptive to implantation. Monitoring of cad-11 distribution and/or expression levels may also be used to determine the optimal time for implantation (the optimal time of endometrial receptivity).

This aspect of the invention therefore provides a method for determining endometrial receptivity to blastocyst implantation comprising determining the level of cadherin-11 production by endometrial cells of a female subject undergoing progestin-increasing therapy and optionally further comprising the step of adjusting said therapy to increase the level of cadherin-11 production, or alternatively optionally further comprising the step of determining the optimal time for blastocyst or implantation. The determination of cadherin-11 production may be compared to a standard as described above.

Yet a further aspect of the invention contemplates the improvement of a woman's fertility by increasing cad-11 expression by endometrium, e.g., using gene therapy. According to this aspect of the invention, cad-11 encoding DNA operably linked to expression control sequences could be delivered or transferred to endometrial cells through DNA constructs such as, e.g., adenoviral vectors. Alternatively, homologous recombination techniques may be used to transfer control of endogenous cad-11 gene expression to different expression control sequences. Also contemplated is use of cad-11 encoding DNA for manufacture of a medicament for increasing cad-11 expression by endometrial cells by delivery of such DNA to endometrial cells.

### DETAILED DESCRIPTION OF THE INVENTION

In this specification, “cad-11” refers to the cadherin of that designation and as described in: Suzuki, S. et al. (1990) “Diversity of the Cadherin Family: Evidence for Eight New Cadherins in Nervous Tissue”, Cell. Regul. 2:261-270; Tanihara, H., et al. (1994) “Cloning of Five Human Cadherins Clarifies Characteristic Features of Cadherin Extracellular Domain and Provides Further Evidence for Two Structurally Different Types of Cadherins”, Cell Adhes. Cozmmn. 2:15-26; and U.S. Pat. No. 5,597,725.

Oligonucleotide sequences which may be used as primers or probes for cad-11 encoding sequences, mRNA or cDNA as described herein are known in the art and have, before this invention, been made and used for those purposes. The sequence information shown in SEQ ID NO:1, or in Tanihara et al., supra, may be readily used to prepare suitable oligonucleotide sequences for use in this invention other than those specifically described in the literature to date.

Production of antibodies to cad-11 and hybridomas producing monoclonal antibodies to cad-11 is described in Example 10 below. Antibodies to cad-11 may also be made according to procedures well established in the art, in particular those procedures described in U.S. Pat. No. 5,597,725 while employing the cDNA sequence information in SEQ ID NO:1, the protein (or an immunogenic fragment of the protein) derived from SEQ ID NO:1 or the corresponding amino acid sequence set out in SEQ ID NO:4, or the sequence information described by Tanihara, et al., supra. Preferred monoclonal antibodies are designated C11-113E and C11-113H, produced, respectively, by hybridomas deposited by ICOS Corporation on Apr. 21, 1998 at the American Type Culture Collection.

One manner in which the method of the invention may be carried out is to test for the presence of a gene encoding cad-11 in any suitable tissue sample of a patient. Absence of such a gene or the presence of mutations would indicate a fundamental inability of the patient to express cad-11 in any tissue and thus would be an indicator of fecundity for that patient. When the gene for cad-11 is mutated or absent, the woman may not be able to establish or maintain a viable pregnancy. In a test for the presence of a gene encoding cad-11 in genomic DNA, it will not matter that a tissue sample (eg. skin, buccal smear, or hair) is one in which cad-11 expression does not occur in the adult. This method includes the hybridization (annealing) to digested genomic DNA of cad-11 cDNA or an oligonucleotide probe corresponding to or homologous to cad-11 cDNA (such as is shown in SEQ ID NO:1, or a fragment thereof) and the detection of hybridized DNA according to procedures well established in the art, including those described in U.S. Pat. No. 5,597,725. Alternatively, mutations in the gene encoding cad-11 may be detected using any method known in the art, including those described in Eng et al., “Genetic testing: The problems and the promise,” Nature Biotechnology, 15:422-426 (1997).

Another aspect of this invention is the detection of cad-11 expression/production in endometrial tissue from a patient. All methods known in the art for the detection of specific RNA or protein from a cellular extract or in a tissue specimen may be employed. For example, any manner of immunological assay employing an anti-cad-11 antibody may be carried out using (where appropriate) a cellular extract or a tissue specimen. Preferably, the antibody will be a monoclonal antibody specific for cad-11. Preferably, the method will involve a suitable detection system whereby binding of the antibody to cad-11 is detected. Any such detection system may be known in the art may be employed, including monitoring the production of an immunoprecipitate, the use of labelled antibodies, electrophoretic separation and detection of stained or labelled antibody-antigen complexes (e.g. Western Blot), antibody sandwich assays, immunohistological assays (including immunochemical, immunofluoresence), and flow cytometry.

The method of this invention employing an anti-cad-11 antibody may be performed on histologically prepared endometrial tissue specimens. Immunohistochemical and immunofluorescent assays are particularly suitable for histological specimens and this methodology permits the detection of cad-11 production associated with different endometrial cells thereby permitting a comparison between epithelial and stromal cells. Procedures for separation of epithelial and stroma cells may be employed prior to other suitable methodologies of this invention involving the preparation of cellular extracts.

The intensity of cad-11 immunostaining in the endometrial biopsies may be determined by the semiquantitative HSCORE technique which is routinely used in oncology for cancer cell counts. The HSCORE is a continuous value (from 0-4, respectively), in which a discriminatory level that designates positives and negatives for the test is determined. Typically, a consensus is made between counts carried out by two observers in a blinded fashion employing a double-headed microscope at low and high magnification.

Cad-11 expression appears as either negative or weak (HSCORE=0-1) or strongly expressed (HSCORE=3-4) in the stroma and epithelium of the secretory endometrium. Thus, in a comparison of the cad-11 production levels of test endometrial biopsy specimens to the levels of standard endometrial biopsy specimens with an HSCORE of 3-4 (eg., from fertile female subjects with the ability to establish and maintain pregnancy, or from fertile female subjects during a time period in which they are receptive to blastocyst implantation), test specimens with an HSCORE of 0-2 indicates that the test subject is not able to establish or maintain pregnancy.

A more defined measurement of cad-11 expression in the epithelial or stromal components of the endometrium may be applied to the test. Under these conditions, the HSCORE would be calculated using the following equation:

ΣPi(i+1) where i=intensity of staining (0=no staining; 1=weak; 2=moderate; and 3=strong staining) and Pi is the percentage of stained epithelial or stromal cells for each intensity, varying from 0% to 100%. In addition, ROC analysis could be applied to the HSCORE measurement. An ROC curve demonstrates the relationship between true-positive ratio and false positive ratios as the definition of a positive test. Computer programs published for this purpose (eg. the SAS program from SAS Institute, N.C., U.S.A.) may be used to determine the optimal HSCORE value to use to predict, for example, LPD.

Statistical methods whereby test samples may be compared to a standard are well-known. For example, results from immunological assays such as ELISA, or results from PCR, may be analyzed and compared to a standard by analysis of variance techniques (ANOVA).

The method of this invention also includes the detection of cad-11 mRNA in cellular extracts of endometrial tissue. Using the known cDNA sequence information for cad-11, all methods of detection of mRNA or recovery of cDNA from mRNA may be employed. For example, Northern Blot analysis of cellular extracts may be performed using cad-11 cDNA as a probe. Likewise, cad-11 cDNA sequence information permits the construction and use of primers whereby cDNA from cad-11 mRNA may be prepared by reverse transcription using established procedures. Such cDNA may be detected, recovered, or amplified by polymerase chain reaction (PCR) and the resulting amplified DNA detected using established procedures. In situ hybridization of a labelled oligonucleotide probe to cad-11 mRNA may also be used in this invention to detect cad-11 mRNA in a tissue or cellular specimen.

The methods of this invention may include one or more of the steps of obtaining a tissue sample from a patient, either preparing a cellular extract from the tissue sample or preparing a histological specimen from the tissue sample, determining the presence of cad-11 or cad-11 mRNA according to the above described procedures and, comparing the results of the determination with results of the same determination as performed using tissue from a known fertile woman or population of known fertile women. The marked difference in endometrial cad-11 production as between fertile women and infertile women or women presenting with habitual abortion or RSA, readily permits a determination as to the inability or ability of a test patient to establish or maintain a pregnancy, and may aid in diagnosing women suffering from habitual abortion or RSA, women suffering from luteal phase deficiency (LPD), and women suffering from unexplained primary infertility.

It is also contemplated that cad-11 may be cleaved and released into the serum of women, and that a determination of serum or plasma levels of cad-11 before or during pregnancy using, eg., an ELISA test can be correlated to the levels of cad-11 expression by endometrium, which are in turn correlated to the woman's ability to establish and maintain a viable pregnancy. Recent studies have indicated that E-cad is cleaved and released into the serum of cancer patients [see Griffiths et al. (1994), Br. J. Cancer, 74:79]. Monitoring of cad-11 levels in serum may be a less invasive test for determining the ability to establish and maintain a pregnancy.

This invention also provides kits for performing the above described methods. The kits may include instructions for their use and information or pictorial displays which permit a comparison to be made between a test patient and the results expected of a fertile woman. The kits may include reagents, mechanical substrates and the like useful in the performance in the above described methods. For example, a kit suitable for an immunohistochemical determination of cad-11 in an endometrial tissue specimen may include the following separate components in containers:

(i) primary antibody (e.g. mouse monoclonal antibody) to human cad-11;

(ii) secondary antibody to monoclonal antibody (e.g. biotinylated horse anti-mouse IgG antibody);

(iii) blocking serum (e.g. horse);

(iv) detectable moiety for secondary antibody (e.g. streptavidin-biotinylated enzyme complex or suitable reagents for this purpose such as Avidin DH solution and biotinylated horseradish peroxidase).

A kit for mRNA determinations will include suitable oligonucleotides which hybridize to cad-11 mRKA and function as reverse transcription (RT) primers or probes. The kit may include standard reagents for reverse transcription including a suitable reverse transcriptase and may include suitable primers and/or enzymes for amplification of cDNA. A kit comprising RT primers for cad-11 CDNA may include only those primers, commercial packaging and instructions and be intended to be used.

Delivery of a functional cad-11 gene to appropriate cells is effected in vivo or ex vivo by use of vectors, and more particularly viral vectors (e.g., Herpes simplex virus, adenovirus, adeno-associated virus, or a retrovirus), or by use of physical DNA transfer methods (e.g., liposomes or chemical treatments). For reviews of gene therapy technology see Friedmann, Science, 244: 1275-1281 (1989); Verma, Scientific American: 68-84 (1990); Miller, Nature, 357: 455-460 (1992); Friedmann, Scientific American, (June, 1997) p. 96-101; and Felgner, Scientific American, (June, 1997) p.102-106. Alternatively, endometrial cells may be modified (e.g., by homologous recombination) to activate the endogenous cad-11 gene that is not being normally expressed or is being expressed at a lower rate than is desired. Suitable modifications can replace, in whole or in part, the naturally-occurring cad-11 promoter with part or all of a heterologous promoter, e.g., in a manner allowing control of cad-11 expression by external means. See, for example, PCT International Publication No. WO 94/12650; PCT International Publication No. WO 92/20808; and PCT International Publication No. WO 91/09955.

Particular embodiments which are illustrative of this invention are described in the following examples. Example 1 examines endometrial cad-11 production (as determined by immunohistochemistry) before and after a specific hormone (progesterone) treatment in women presenting with habitual abortion associated with luteal phase deficiency (LPD) Example 2 examines endometrial cad-11 production (as determined by immunohistochemistry) in women with primary infertility either unexplained or in association with LPD. Example 3 describes a protocol for performing immunohistochemistry detection of cad-11 production in endometrial biopsy specimens. Example 4 examines endometrial cad-11 production in normal women during the menstrual cycle and in cell cultures treated with various gonadal steroids. Example 5 describes a protocol for performing Northern blot analysis to determine levels of cad-11 mRNA transcripts. Example 6 describes a protocol for performing Western blot analysis to detect levels of cad-11 production. Example 7 describes a protocol for performing flow cytometry to detect the presence of cad-11 on the surface of endometrial cells. Example 8 describes a protocol for performing reverse transcriptase polymerase chain reaction to produce cad-11 cDNA. Example 9 describes a protocol for performing in situ mRNA hybridization to detect cad-11 mRNA transcripts. Example 10 describes preparation of hybridomas producing monoclonal antibodies that bind to cadherin-11. Example 11 describes production of a cad-11 expression vector and a recombinant Adenovirus vector.

### EXAMPLE 1

### Cadherin-11 Expression in Women Presenting with Hormonal-Associated, Habitual Abortion

Habitual abortion in this study is defined as three or more consecutive spontaneous abortions. To date histological dating of endometrial biopsies, is considered to be the most reliable method of determining the likelihood of implantation and early pregnancy. However, the considerable variation between pathologists and the low accuracy of endometrial dating suggest that this technique is no longer acceptable. Cad-11 expression/production is a superior marker.

The presence of cad-11 was examined in endometrial biopsies obtained from women presenting with primary (no prior live birth) habitual abortion in association with luteal phase deficiency (LPD). This group of women, who account for approximately 20% of the women attending the Recurrent Pregnancy Loss Clinic, B. C. Women's Hospital and Health Centre, University of British Columbia, Vancouver, Canada, are routinely treated with progesterone or clomiphene citrate (a nonsteroidal synthetic compound). Biopsy specimens were obtained from these women before and after treatment. Cad-11 expression/production was correlated with pregnancy outcome upon treatment. Present treatment of LPD consists of either vaginal progesterone suppositories or clomiphene citrate, with the dosage being adjusted until a repeat endometrial biopsy is considered to be “in phase” by morphological assessment.

LPD was diagnosed by traditional methods prior to determining cad-11 by immunostaining. To increase the likelihood of including only those individuals with authentic LPD, the criteria for inclusion in this study was two consecutive biopsies with ≧3 days of maturation delay based on the first day of the next menses as determined by morphological assessment.

In 16 such patients, cad-11 immunostaining was examined in biopsies obtained before and after treatment with either progesterone (results shown in Table 1A) or clomiphene citrate (results shown in Table 1B). All 16 had return of normal in-phase morphology on subsequent biopsy. The endometrial biopsy specimens were then re-evaluated for cad-11 using immunohistochemical staining. Comparison of staining pattern and intensity was performed using a monoclonal antibody directed against human cadherin-11 and standard immunohistochemical techniques according to the protocol set out in Example 3. The intensity of cad-11 immunostaining in the endometrial biopsies was determined by the semiquantitative HSCORE technique as described above.

As is shown in the results set out in Tables 1A and 1B, cad-11 was not detected in the glandular and luminal epithelium of the endometrium of the women prior to treatment. These patterns are in direct contrast to those observed in fertile women who have cad-11 in the luminal and glandular epithelium at all stages of the menstrual cycle. Furthermore, cad-11 was not detected in the stroma of the endometrial biopsies in the mid-late secretory phase, again in contrast to the pattern observed in fertile women.

Following treatment with either clomiphene citrate or progesterone, the endometrium of these women were found to be normal and in-phase, as determined by routine morphological assessment. However, different intensities of cad-11 immunostaining in the epithelial and stromal component of the endometrium observed in this cohort of women (HSCORES=0-4: Tables 1A and 1B). This shows that both progesterone and clomiphene citrate are capable of increasing endometrial cad-11 expression, although the degree of response is variable. Correlation of cad-11 is production with pregnancy outcome in this cohort of women revealed that the most intense cad-11 immunostaining (HSCORES=3-4) was observed in women who maintained a viable pregnancy. Thus, a positive HSCORE (3-4) for endometrial cad-11 expression in a treated cycle was observed to correlate with a successful pregnancy outcome in women with LPD-associated habitual abortion. Conversely, a negative HSCORE (0-1) following treatment correlated with a subsequent pregnancy loss (spontaneous abortion).

TABLE 1A
CAD-11 EXPRESSION IN THE ENDOMETRIUM OF WOMEN
PRESENTING WITH PRIMARY HABITUAL ABORTION
AND DIAGNOSED WITH LUTEAL PHASE DEFICIENCY
Treatment: Progesterone Suppositories

| | BEFORE | | |
|---|---|---|---|
| | TREATMENT | AFTER TREATMENT | PREGNANCY |
| PATIENT | (HSCORE) | (HSCORE) | OUTCOME |
| 1 | 0 (negative) | 3 (positive) | successful |
| 2 | 0 (negative) | 3 (positive) | successful |
| 3 | 0 (negative) | 4 (positive) | successful |
| 4 | 0 (negative) | 4 (positive) | successful |
| 5 | +1 (negative) | 2 (equivocal) | spontaneous |
| | (glands only) | | abortion |
| 6 | 0 (negative) | 1 (negative) | spontaneous |
| | | | abortion |
| 7 | 0 (negative) | 0 (negative) | spontaneous |
| | | | abortion |
| 8 | 0 (negative) | 1 (negative) | spontaneous |
| | | | abortion |

TABLE 1B
Treatment: Clomiphene Citrate

| | BEFORE | | |
|---|---|---|---|
| | TREATMENT | AFTER TREATMENT | PREGNANCY |
| PATIENT | (HSCORE) | (HSCORE) | OUTCOME |
| 1 | 0 (negative) | 3 (positive) | successful |
| 2 | 0 (negative) | 3 (positive) | successful |
| 3 | 0 (negative) | 4 (positive) | successful |
| 4 | 0 (negative) | 4 (positive) | successful |
| 5 | 0 (negative) | 2 (equivocal) | spontaneous |
| | | | abortion |
| 6 | 0 (negative) | 1 (negative) | spontaneous |
| | | | abortion |
| 7 | 0 (negative) | 0 (negative) | spontaneous |
| | | | abortion |
| 8 | 0 (negative) | 1 (negative) | spontaneous |
| | | | abortion |

### EXAMPLE 2

### Cad-11 Expression in Women Presenting with Primary Infertility

Diminished endometrial receptivity resulting in failed or defective implantation is a mechanism that may account for much infertility that is not related to anovulation, tubal obstruction or poor semen quality. Luteal phase deficiency (LPD), a disorder characterized by delayed maturation of the endometrium during the secretory phase of the menstrual cycle, is associated with infertility and habitual abortion as a consequence of dyssynchronous embryonic and endometrial development. This disorder may be only one of many causes of an unreceptive endometrium as even when morphological development of the endometrium proceeds normally, as is seen in patients with unexplained infertility, its functional maturation may be delayed or otherwise impaired. In addition, despite increasing experience with assisted reproductive technologies, only a low proportion of women undergoing in vitro fertilization and embryo transfer establish a viable pregnancy. A limiting factor in this setting may also be the ability of the embryo to attach and subsequently invade into the endometrium. Cad-11 is a useful marker for luteal phase maturation and endometrial receptivity since cad-11 is not expressed in the endometrium of women with primary infertility (no prior live birth), either unexplained or in association with LPD.

The presence of cad-11 was examined in endometrial biopsies obtained from primary infertile patients with documented LPD. An LPD was defined as an endometrial biopsy with a ≧3 days of maturation delay, based on the day of ovulation and morphological assessment. Endometrial tissues obtained from fertile control subjects who sought elective sterilization or had documented male infertility were used as controls. These tissues were morphologically dated and matched to the timing of the endometrial biopsies obtained from the LPD patients (days 20-24 of the menstrual cycle). Six matched endometrial biopsies were subsequently used in the study. After determining that cad-11 immunostaining is an accurate predictor of endometrial dysfunction in the subpopulation of women with LPD, the ability of cad-11 to determine endometrial dysfunction in women with unexplained infertility was assessed. Cad-11 in the endometrial biopsies was determined by immunohistochemistry using a monoclonal antibody directed against cadherin-11 and standard techniques according to the protocol set out in Example 3.

Cad-11 immunostaining in the endometrial biopsies was quantified by the semiquantitative HSCORE technique as described above. As shown in Table 2A, cad-11 (HSCORE=0) was not found in either the glandular epithelium or stroma of secretory endometrium obtained from a cohort of six women presenting with primary infertility diagnosed with LPD. These results are in contrast to the expression pattern of cad-11 in the dated endometrial biopsies of women with proven fecundity (HSCORE=3-4). Low levels of cad-11 were detected in a further three women diagnosed with unexplained primary infertility (results for glands shown in Table 2B).

TABLE 2A
CAD-11 EXPRESSION IN THE ENDOMETRIUM OF WOMEN
PRESENTING WITH PRIMARY INFERTILITY
DIAGNOSED WITH LUTEAL PHASE DEFICIENCY

| | FIRST BIOPSY | SECOND BIOPSY |
|---|---|---|
| PATIENT | (HSCORE) | (HSCORE) |
| 1 | 0 | 0 |
| Normal Control | Glands 4, Stroma 3 | Glands 4, Stroma 3 |
| 2 | 0 | 1 |
| Normal Control | Glands 4, Stroma 3 | Glands 4, Stroma 4 |
| 3 | 1 | 0 |
| Normal Control | Glands 4, Stroma 4 | Glands 4, Stroma 4 |
| 4 | 0 | 0 |
| Normal Control | Glands 4, Stroma 3 | Glands 4, Stroma 3 |
| 5 | 1 | 0 |
| Normal Control | Glands 4, Stroma 3 | Glands 4, Stroma 4 |
| 6 | 0 | 1 |
| Normal Control | Glands 3, Stroma 3 | Glands 4, Stroma 4 |

TABLE 2B
CAD-11 EXPRESSION IN THE ENDOMETRIUM OF WOMEN
PRESENTING WITH PRIMARY UNEXPLAINED INFERTILITY

| | FIRST BIOPSY | SECOND BIOPSY |
|---|---|---|
| PATIENT | (HSCORE) | (HSCORE) |
| 1 | 0 | 0 |
| 2 | 0 | 1 |
| 3 | 2 | 1 |

### EXAMPLE 3

### Strept-avidin Horseradish Peroxidase Immunohistochemistry Protocol

For Detection of Cad-11 in Endometrial Biopsy Specimens

Prepare diluent fresh 1% bovine serum albumin (BSA; Sigma Chem Co.)/1% Automation Buffer (AB; ESBE Lab.) (wt/vol).

For paraffin embedded endometrial biopsy specimen sections (5 μm):

1. Wash tissue specimen thoroughly in phosphate-buffered saline (PBS).

2. Fix in 4% paraformaldehyde for 4-12 hours.

3. Wash in PBS (2 changes, 5 min).

4. Dehydrate in graded series of ethanol (30, 50, 70, 80, 90, 100, 100%, 30 min each).

5. Clear in 100% xylene (2 changes, 30 min each).

6. Embed tissue in paraffin (Paraplast™; 2 changes, 60 min. 58° C.).

7. Cut sections, transfer to glass microscope slide (coated in 1% BSA) and proceed to deparaffinisation step.

For frozen endometrial biopsy sections (5 μm):

1. Wash tissue specimen thoroughly in phosphate-buffered saline (PBS).

2. Embed in O.C.T. compound (Miles Inc.) and snap-freeze in liquid nitrogen.

3. Cut sections and transfer to glass microscope slide (coated in 1% BSA).

4. Fix tissue in 4% paraformaldehyde for 30 min.

5. Wash in tap water (2 changes, 5 min.).

6. Air dry sections and store at −70 C or proceed immediately to endogenous peroxidase block.

Deparaffinisation:

1. Incubate slides in 100% Xylene (3 changes, 5 min each, room temperature).

2. Fix sections in absolute ethanol (3 changes, 2 min each, room temperature).

3. Wash in running tap water (5 min).

Endogenous Peroxidase Activity Block:

Prepare a fresh solution of methanol 2% hydrogen peroxide (vol/vol) using a 30% stock solution of hydrogen peroxide (BDH Chem).

1. Incubate slides in methanol/2% hydrogen peroxide solution for 20 min at room temperature.

2. Wash in running tap water for 5 min.

Normal Horse Serum Non-Specific Block:

Prepare a 10% solution of Normal Horse Serum (NHS; Vector Lab.)/1% BSA/1×AB (vol/vol).

1. Wash slides in 1% BSA/1×AB (2 changes, 5 min each, room temperature).

2. Wipe off excess buffer from glass microscope slide.

3. Incubate tissue sections with 10% NHS (20 min, 37° C.) in a humidified chamber.

Primary Antibody:

The cadherin-11 (cad-11) antibody (C11-113E; ICOS Corporation) is a mouse monoclonal IgG generated against human cad-11 peptide fragments as described in Example 10 below. The antibody is used at 1:200 dilution (diluted in 1% BSA/1×AB). Primary antibody is omitted for technical control sections.

1. Drain excess NHS blocking reagent and wipe excess fluid from the slide.

2. Incubate sections with primary antisera (45 min, 37° C.) in a humidified chamber.

3. Wash slides in 1% BSA/1×AB (2 changes, 5 min each, room temperature.

Secondary Antibody:

Biotinylated horse anti-mouse IgG (Vector Lab) is used at a 1:200 dilution.

1. Drain excess diluent and wipe excess fluid from the slide.

2. Incubate sections with secondary antisera (30 min. 37° C.) in a humidified chamber.

3. Wash slides in 1% BSA/1×AB (2 changes, 5 min each, room temperature).

Biotinylated Strept-Avidin Horse Radish Peroxidase Detection Reagent:

Use DAKO Corp.'s StreptABC™ complex at a final dilution of 1:50. Prepare a 1:100 dilution of strept-avidin solution (Sol. A) and mix with a 1:100 dilution of biotinylated horseradish peroxidase (Sol. B). StreptABC™ complex reagent should be prepared at least 30 min prior to use. Save a drop of StreptABC™ complex for chromogen detection test.

1. Drain excess diluent and wipe excess fluid from the slide.

2. Incubate sections with StreptABC complex (30 min, 37° C.) in a humidified chamber.

3. Wash slides in 1% BSA/1×AB (2 changes, 5 min each, room temperature.

Chromogen Detection:

Use 3,3′-diaminobenzidine (DAB) (Sigma Chem. Co.,) for chromogen detection. Prepare a 0.05% DAB/diluent solution (wt/vol) at least 30 min prior to use. Immediately before use, add 0.1% (vol/vol) of 30% stock hydrogen peroxide to the DAB solution. Test DAB solution by adding a drop of StreptABC complex to a drop of DAB/hydrogen peroxide solution. If a brown colour is detected in the test solution, proceed with chromogen detection.

1. Incubate slides with fresh DAB/hydrogen peroxide solution (5 min, room temperature.

2. Wash in running tap water for 2 min.

Counterstaining, Clearing and Mounting:

1. Incubate slides in Harris' haematoxylin (BDH Chem) for 30 seconds (sec).

2. Wash in running water for 2 min.

3. Decolorise sections in 4% glacial acetic acid (BDH Chem) (vol/vol) for 20 sec.

4. Wash in distilled water for 2 min.

5. Sections are turned blue in 1% lithium carbonate (Fischer Sci.) solution (wt/vol) for 30 sec.

6. Wash in distilled water.

7. Dehydrate slides in absolute ethanol (3 changes, 2 min each, room temperature).

8. Clear slides in 100% xylene (3 changes, 2 min each, room temperature).

9. Mount slides in synthetic mounting media (Cytoseal 60™; Stephens Sci.) by adding a drop of mounting media to the microscope slide and placing a glass coverslip on top of the section.

### EXAMPLE 4

### Detection of Cad-11 mRNA Transcripts in the Human Endometrium In Response to Hormone Treatment

A. Cad-11 Production by Normal Glandular and Stromal Epithelium

Human endometrial tissue biopsies were obtained from women of reproductive age. All patients had normal menstrual cycles and had not received hormones for a least 3 months prior to the collection of tissue. The stage of the menstrual cycle was determined by the next menstrual period and confirmed by histological evaluation. Tissues used in this study were obtained between the midproliferative (day 6) and the late secretory phase (day 28) of the menstrual cycle.

The epithelial and stromal components of proliferative and secretory endometrium were separated by enzymatic digestion and mechanical dissociation. The endometrium was minced and subjected to collagenous digestion (0.25%) at 37° C. for 30 min. The stroma cells were isolated from the epithelial cells by passing the supernatant through a sieve is (40 μm). The isolated glands were retained on the sieve. The stroma cells were collected in a 50 ml tube and purified by layering the supernatant on a Ficoll-Paque gradient and centrifuging the columns at 400×g for 10 min.

Isolated glandular epithelium and stroma cells prepared as described above were immediately harvested for Northern Blot analysis as described in Example 5. Blots were probed with a human cad-11 cDNA (1.6 kb) obtained from a human placental cDNA library which contained nucleotides 1095-2620 of SEQ ID NO:1.

A single cad-11 mRNA transcript of 4.4 kb was detected in the glandular epithelium at all stages of the menstrual cycle. However, cad-11 mRNA transcripts were not found in the endometrial stroma until the mid-secretory phase of the menstrual cycle. Stroma cad-11 mRNA levels continued to increase as the cycle entered the late secretory phase.

B. Effect of Progesterone or Estradiol on Stromal Cells in Culture

Endometrial stroma cells were isolated from secretory endometrium as described above. The stroma cells were grown to confluence, washed with PBS, and cultured in DMEM containing charcoal stripped FCS for a further 24 h. The culture medium was removed, and after the cells had been washed twice with PBS, replaced with fresh DMEM containing charcoal stripped FCS. The cells were harvested for either Northern (see Example 5) or Western blot analysis (see Example 6) after 24 h of culture in the presence or absence of steroids in the culture medium P4 (0.1-1 μM), E2 (30 nm), or vehicle (0.1% ethanol).

Progesterone (P4) increased cad-11 mRNA levels in a dose-dependent manner as determined by Northern blot analysis using the same cad-11 cDNA probe as in Example 4A above. Western blot analysis using extracts prepared from stroma cells treated with P4 and a mouse monoclonal antibody directed against human cad-11 revealed a single cad-11 protein species (125 kDa). In agreement with the Northern blot analysis, P4 treatment induced an increase in stromal cad-11 protein. In contrast, 17β-estradiol (E2) had done little effect on cad-11 mRNA levels.

C. Effect of Gonadal Steroids on Stromal Cad-11 mRNA and Protein Over Time

Endometrial stromal cells were separated from the glandular epithelium by enzymatic digestion and mechanical dissociation as described above. The endometrial stromal cells were washed once in phenol red-free Dulbecco's Modified Eagle's medium (DMEM) containing 10% charcoal-stripped fetal bovine serum (FBS) before being resuspended and plated in DMEM containing 25 mM glucose, 25 mM Hepes, 1% (w/v) L-glutamine, antibiotics (100 U/ml endometrial stromal cells. The addition of vehicle (0.1% ethanol) to the culture medium had no effect on the levels of the cad-11 mRNA transcript present in these endometrial stromal cell cultures. In contract, P4 caused a significant increase in the stromal cad-11 mRNA levels after 24 h of culture in the presence of this steroid. The levels of the cad-11 mRNA transcript continued to increase until the duration of these studies at 96 h. E2, or DHT alone did not significantly increase cad-11 mRNA levels at any of the time points examined in these studies.

Western blot analysis, using extracts prepared from endometrial stromal cells cultured in the presence of gonadal steroids and a mouse monoclonal antibody directed against human cad-11, revealed a single cad-11 protein species (125 kDa) in all of the cellular extracts. In agreement with the Northern blot analysis, P4 caused an increase in cad-11 expression after 24 h of culture in the presence of this steroid. The expression levels of cad-11 continued to increase until the duration of these experiments at 86 h. No significant increase in cad-11 expression levels in endometrial stromal cells in the presence of vehicle, E2, or DHT at any of the time points was detected in these studies.

D. The Effects of P4 Plus E2 or DHT on Cad-11 mRNA and Protein Level in Endometrial Stromal Cells

To determine whether a combination of steroids was required for maximal cad-11 expression in endometrial stromal cells, the cells (prepared as described above) were cultured in the presence of P4 (1 μM) plus E2 (30 nM), or P4 (1 μM) plus DHT (0.1 μM) for 0, 6, 12, 24, 48, 72 or 96 h before being harvested for Northern or Western blot analysis as described above.

There was a significant increase in cad-11 mRNA and protein expression levels in endometrial stromal cells cultured in the presence of E2 plus P4 for 12 h. Similarly, stromal cad-11 protein expression levels were significantly increased after 12 h of culture under these conditions. Cad-11 mRNA and protein expression levels continued to increase until the duration of these studies at 96 h. The cad-11 mRNA and protein levels detected in the endometrial stromal cells cultured in the presence of E2 plus P4 for 12-96 h were significantly greater than those observed in cells cultured in P4 for the same periods of time. In contrast, there was no significant difference between the cad-11 mRNA and protein levels observed in cells cultured in the presence of P4 plus DHT and those detected in cells cultured in P4 alone at any time points examined in these studies (p<0.05).

E. The Effects of Varying Doses of E2 to Potentiate the P4-Mediated Increase in Stromal Cad-11 mRNA and Protein Levels

To determine whether the ability of E2 to potentiate the effects of P4 on stromal cad-11 expression was dose-dependent, the cells were cultured in the presence of vehicle (0.1% ethanol), E2 (30 nM), P4 (1 μM) or P4 (1 μM) plus varying doses of E2 (0.5, 1.0, 5.0, 10.0, 30+100 nM) for 96 h. 30 nM is approximately equivalent to physiological levels. The cells were then harvested for Northern and Western blot analysis using the procedures described above.

Increasing doses of E2 progressively enhanced the effects of P4 on stromal cad-11 mRNA and protein levels. Maximum cad-11 mRNA and protein expression level were observed in cells cultured in the presence of 30 nM E2. There was no further enhancement in stromal cad-11 mRNA and protein expression levels when the concentration of E2 was increased to 100 nM. Thus, progestin treatment will be optimized in an estrogen environment but near optimal effect on cad-11 production in the endometrium should be achieved in patients with normal physiological levels of estrogen.

F. Gonadal Steroids Shown to Regulate Cadherin-11 mRNA and Protein Expression Levels in Cultured Endometrial Stromal Cells

The following progestins increased cad-11 expression as determined by procedures as described above:

Pregnenolone

Progesterone

medroxyprogesterone acetate

Thus, progestins as a group are capable of regulating cad-11 mRNA and expression levels in endometrial stromal cells. Drugs, such as clomiphene, which increase progestin production in a patent are also effective.

Estrogen will potentiate the effect of progestins. 17β-estradiol and estrone both potentiate this effect, but not 17a-estradiol which has no known biological activity. Androgens per se, are incapable of regulating cad-11 mRNA and protein expression levels in cultured endometrial stromal cells or potentiating the P4-mediated increase in the expression of this stromal cell adhesion molecule. However, testosterone which can be converted into estrogen (by the enzyme aromatase), will potentiate progestin-mediated effects but not DHT which is incapable of conversion to estrogen by aromatase.

### EXAMPLE 5

### Protocol For Northern Blot Analysis

RNA Extraction using an RNAID™ kit (BIO 101, Inc.):

1. 500 μl cell solution+500 μl of 2M sodium acetate (pH 4.0).

2. Vortex.

3. Add 525 μl of chloroform/isoamyl alcohol.

4. Vortex.

5. Incubate on ice for 15 minutes.

6. Spin at 4° C./10,000×g for 20 minutes.

7. Transfer top phase (RNA rich portion) to a new vial, taking care not to touch the interface and bottom organic solvent part.

8. Add ½ volume of chloroform/isoamyl alcohol.

9. Vortex.

10. Spin 2 min at 4° C./10,000×g.

11. Transfer top phase to a new tube.

12. Add 20 μl of RNA Matrix.

13. Vortex 30 sec.

14. Incubate at room temperature for 5 min, with occasional mixing.

15. Spin 1 min at top speed of microcentrifuge to pellet the RNA/RNA Matrix complex.

16. Transfer the supernatant to a new tube for reabsorption if necessary.

17. Respin the pellet briefly and remove the traces of liquid.

18. Re-suspend the white pellet in 300 μl of RNA binding salt, stir with pipet tip.

19. Spin 1 min at top speed, and save the supernatant for reabsorption if necessary.

20. Re-suspend the pellet in 500 μl of RNA Wash solution (add ethanol before first use).

21. Mix with pipet tip.

22. Spin 1 min at top speed, remove and save the supernatant in a new tube.

23. Re-suspend the pellet in 500 μl of RNA Wash solution again.

24. Spin 1 min at top speed, remove and save the supernatant in a new tube.

| | |
|---|---|
| 1 ml | 100 × Denhardts solution |
| 5 ml | 20χ SSPE |
| 0.5 ml | 40χ NaPO₄ |
| 1.6 ml | 60% dextran sulphate |
| 0.7 ml | H₂O |
| 1 ml | 20% SDS |
| 0.2 ml | 20 mg/ml stock salmon sperm DNA (denature) |

15. Remove the BSA/SSPE solution and replace with hybridization solution.

16. Incubate at 42° C. for one hour with agitation.

17. Denature the p³² labelled cDNA and add 2,000,000 CPM per ml to the hybridization solution and incubate at 42 C overnight with agitation.

Preparation of Formaldehyde-Agarose Gels:

1. For 100 ml: lg agarose

10 ml 10×MOPS

80 ml RNAse-free water

10 ml 37% Formaldehyde.

2. Resuspend RNA in 50-100 μl the following solution:

0.72 ml formamide

0.16 ml 10×MOPS

0.18 ml RNAse-free Water

0.1 ml 80% glycerol

0.08 ml bromphenol blue and cyanol (saturated solution).

3. Load total RNA samples (20-25 μg) into the wells of the gel and run the gel submerged in 1×MOPS solution at 45 V.

4. The RNA transcripts which are electropheretically separated by size are transferred from the gel onto a nylon membrane (pre-soaked in 10×SSC) by vacuum manifold for 60 min.

5. The nylon membrane is allowed to dry at room temperature before being probed with radiolabelled cDNA probes.

Washing the Membrane:

1. Remove the radioactive hybridization solution.

2. Wash in 20-30 ml of 2×SSPE for 5 minutes at room temperature.

3. Repeat step 2.

4. Wash in 20-30 ml of 2×SSPE/1% SDS, at 55° C. for 30 minutes.

5. Repeat step 4.

6. Wash in 20-30 ml of 0.2×SSPE at room temperature for 30 minutes.

7. Repeat step 6.

8. Drain membrane and pack with X-ray film for 24 hours.

9. Develop film.

### EXAMPLE 6

### Protocol for Western Blot Analysis of Protein In Cellular Extracts from Endometrial Tissue

Cell cultures were rinsed three times with PBS and drained. The cells were incubated in 100 μl of cell lysis buffer (Tris HCL, pH 7.5 containing 0.5% NP-40, 0.5 mM CaCl₂, and 1.0 mM PMSF) at 4° C. for 30 min on a rocking platform. Cell lysates were collected by scraping plate with a plastic spatula. The cell lysates were centrifuged at 10,000×g for 20 min, and the supernatant was used in the Western Blot analyses or stored at −70° C. Aliquots from the samples were subjected to SDS polyacrylamide gel electrophoresis under reducing conditions. The stacking gels contained 5% acrylamide, and the separating gels were composed of 7.5% acrylamide. The proteins were electrophoretically transferred from the gels onto nitrocellulose membrane. The nitrocellulose blots were 5 μl of an optimal dilution of PE mouse anti-human 5B5 monoclonal antibodies is added to the other three test tubes in each group.

To identify the presence of cadherin-11 on the surface of either the glandular or stromal cells, 100 μl of an optimal dilution of fluorescein isothiocyanate-conjugated (FITC) F(ab′)₂ fragments of mouse anti-human cadherin monoclonal antibodies prepared using an IMMUNOPURE™ Fab preparation kit (Pierce Chemicals), is added to the four test tubes.

The twelve tubes are then incubated for 30 min at 4° C. The cells are twice washed with 2 mL of PBS containing 0.1% NaN₃ and 1% FCS followed by centrifugation, at 400×g at room temperature for 10 min. The supernatant is decanted leaving a dry cell pellet at the bottom of each test tube. The cells are fixed in 0.3 mL of it paraformaldehyde.

The samples are analyzed using an EPICS Profile I™ (Courter Electronics, Miami, Fla.) flow cytometer, equipped with a 15 mW argon laser (488 nm excitation, 250 mW emission). The argon laser is aligned with DNA-Check™ (Courter Corp., Miami, Fla.) beads. Fluorescence is standardized using Standard Brite™ (Courter Corp., Miami, Fla.) beads. Data is collected with logarithmic amplification, and fluorescence intensity is displayed on a 256-channels, four decade log scale.

For each test tube, an electronic gate (bitmap) is manually drawn around the sample population, based on their forward scatter and side scatter properties. Phycoerythrin (PE) fluorescence of 10,000 cells from within the bit map is plotted on a single parameter histogram, on a 256 channel, four decade log scale. A window gate is drawn across the PE positive cell population. Fluorescein isothiocyanate (FITC) fluorescence of cells within the PE window gate is plotted to a single parameter histogram, on a 256 channel, four decade log scale. A cursor is drawn across the x-axis to determine the logarithmic mean channel FITC fluorescence. Using the table supplied by the manufacturer, the logarithmic mean channel fluorescence is converted to the linear mean channel fluorescence. Channel shifts are completed by subtracting the linear mean channel fluorescence of the negative control from the test sample.

### EXAMPLE 8

### Reverse Transcriptase-Polymerase Chain Reaction (RT/PCR) Protocol

Total RNA is extracted from isolated endometrial cell types from endometrial biopsies as described in the preceding examples. Two oligonucleotides which are specific for cadherin-11 which may be used for RT/PCR are:

| | |
|---|---|
| Forward primer: | |
| 5′-CTCCTCCGTATTACTCCATTCAA-3′ | (SEQ. ID. NO:2) |
| Reverse primer: | |
| 5′-ATTTGCTCCAGGTGTCAAGACAT-3′. | (SEQ. ID. NO:3) |

Reverse Transcription:

1. 2 μl 10×Enzyme Buffer.

2. 2 μl dNTPs (25 mM each).

3. 4 μl Magnesium chloride (50 mM).

4. 7 μl RNAse-free water.

5. 2 μl Reverse primer (50 μM).

6. 1 μl RNAse inhibitor.

7. 1 μl M-MLV Reverse transcriptase.

8. 1 μg Total RNA.

Incubate:

15 min at 42° C.

5 min at 95° C.

5 min at 4° C.

Polymerase Chain Reaction:

1. 8 μl 10×Enzyme buffer.

2. 4 μl Magnesium chloride (50 mM).

3. 65.5 μl RNAse-free water.

4. 2 μl Forward primer (50 μM).

5. 0.5 μl Taq DNA polymerase.

Layer 40 μl of mineral oil on top.

Cycling Program:

The cycling program is repeated 35 times:

95° C. for 1 min

65° C. for 1.5 min

72° C. for 3 min

### EXAMPLE 9

### Protocol for In situ Hybridization to Cellular RNA Using Paraffin or Frozen Tissue Sections

Dewaxing and Rehydration:

1. Three changes in xylene, 2 mins each.

2. Rehydrate in:

100% ethanol-twice, 2 min each

95% ethanol-2 min

70% ethanol-2 min

50% ethanol-2 min.

Denaturation:

1. Denature specimens 20 min at room temperature in 0.2 N HCl.

2. Heat denature 15 min at 70° C. in 2×SSC.

3. Rinse 2 min in 1×PBS.

4. Post fix specimens at 5 min at room temp in 4% paraformaldehyde.

5. Block fixation 5 min in 3×PBS.

6. Rinse twice, 30 sec each time in 1×PBS.

Blocking:

1. Equilibrate specimens in 10 mM DTT prepared in 1×PBS for 10 min at 45° C. in water bath.

2. Block specimens 1× PBS containing 0.167 g DTT, 0.74 g iodoacetamide, and 0.5 g N-ethylmaleimide for 30 min at 45° C. Cover with aluminum foil.

3. Rinse twice, 2 min each time, in 1×PBS at room temperature.

4. Equilibrate specimens 2 min in freshly prepared TEA buffer (0.1 M triethanolamine Cl pH 8.0).

5. Transfer slides to fresh TEA buffer and add acetic anhydride to a concentration of 0.25%. Mix quickly and incubate slides for 5 min with agitation. Add additional acetic anhydride to reach a final concentration of 0.5% and incubate for a further 5 min.

6. Block specimens 5 mins in 2×SSC.

Dehydrate the Specimens:

1. Dehydrate in

50% ethanol—2 min

70% ethanol—2 min

95% ethanol—2 min

100% ethanol (twice)—2 min.

2. Air dry specimens and store at −70° C. overnight.

Synthesis and Preparation of S³⁵-labelled cad-11 cDNA probe:

Radiolabelled cad-11 cDNA (e.g. 1.6 kb insert) is prepared by random primer extension using [S³⁵] dNTPs. Add 10 mM DTT to the standard reaction mixture (see below) containing two different [S³⁵] dNTPs (4 μM). Incubate at 37° C. for at least 30 min.

Reaction mix:

4 μl 5×enzyme buffer

0.2 μl 1M DTT

1.0μ of two of the 10 mM NTPs

1 μg denatured cDNA

10 μg [35S] dNTPs

16 U DNA polymerase.

Denature the probe at 95° C. for 5 mins, 4° C. for 5 min.

Immediately add enough hybridization buffer to obtain 0.3 μg/ml final probe concentration. Mix well and count 1 μl (expected counts>1×10⁵ cpm/μl). Place tubes in water bath at 45° C.

Hybridization Buffer:

50% formamide

0.3 M NacCl

10 mM Tris-HCl, pH 8.0

1 mM EDTA

1× Denhardt's solution

500 μg sheared salmon sperm

50 mM DTT

10% polyethylene glycol

Coat slides with hybridization buffer and place in nmoist incubation chamber for a 30 min-4 h period, at 45° C.

Washing:

Wash slides in:

1) 50% formamide

2×SSC

20 mM B-mercaptoethanol

for 15 min at 55° C., twice;

2) 50% formamide

2×SSC

20 mM B-mercaptoethanol

0.1% Triton X-100

for 15 min at 55° C., twice;

3) 2×SSC

20 mM B-mercaptoethanol

for 15 min at room temperature, twice.

RNAse Digestion:

1. Add 500 μl RNAse digestion solution:

40 μg/ml RNAse A

2 μg/ml RNAse Tl

10 mM Tris HCL, ph 7.5/5 mM EDTA

0.3 M NaCl.

Incubate slides in moist chamber for 15 min.

2. Wash slides twice at 50° C. with agitation, 30 min each time, in:

2×SSC

20 mM B-mercaptoethanol.

3. Wash slides twice at 50° C. with agitation, 30 min each time, in:

50% formamide

2×SSC

20 mM B-mercaptoethanol.

4. Wash slides twice, 5 min each time, in 2×SSC.

5. Dehydrate in

50% ethanol/0.3 M ammonium acetate—2 min

70% ethanol/0.3 M ammonium acetate—2 min

95% ethanol/0.3 M ammonium acetate—2 min

100% ethanol (twice)—2 min.

6. Air dry slides.

7. Expose slides at least overnight with Du Pont Kronex™ Video Imaging Film (MRF-Clear) at 4° C. under light pressure.

### EXAMPLE 10

### Production of Cadherin-11 Monoclonal Antibodies By ICOS Corporation

A mouse was injected three times at three-week intervals with a fusion protein consisting of domains 1-3 of cad-11 fused to maltose binding protein, designated C11/MBP. The C11/MBP fusion protein was prepared as described in Example 4 of U.S. Pat. No. 5,597,725, incorporated herein by reference. Four weeks later, the mouse was given a prefusion boost of CD11/MBP in PBS. Three days later, the mouse was sacrificed and its spleen removed. A single-cell suspension was formed by grinding the spleen between the frosted ends of two glass microscope slides submerged in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspension was filtered through sterile 70-mesh Nitex cell strainer (Becton Dickinson, Parsippany, N.J.), and washed twice by centrifuging at 200 g for 5 minutes and resuspending the pellet in 20 ml serum-free RPMI. Thymocytes taken from 3 naive Balb/c mice were prepared in a similar manner. NS-1 myeloma cells, kept in log phase in RPMI with 11% FetalClone serum (FBS) (Hyclone Laboratories, Inc., Logan, Utah) for three days prior to fusion, were centrifuged at 200 g for 5 minutes, and the pellet was washed twice as described in the foregoing paragraph. After washing, each cell suspension brought to a final volume of 10 ml in serum-free RPMI, and counted.

Spleen cells were combined with NS-1 cells in a ratio of 5:1, centrifuged and the supernatant was aspirated. The cell pellet was dislodged by tapping the tube and 2 ml of 37° C. PEG 1500 (50% in 75 mM Hepes, pH 8.0) (Boehringer Mannheim) was added with stirring over the course of 1 minute, followed by adding 14 ml of serum-free RPMI over 7 minutes. An additional 16 ml RPMI was added and the cells were centrifuged at 200 g for 10 minutes. After discarding the supernatant, the pellet was resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×10⁶ thymocytes/ml. The suspension was dispensed into ten 96-well flat bottom tissue culture plates (Corning, United Kingdom) at 200 μl/well.

On days 3, 5, 7, and 8 after the fusion, 100 ul of medium was removed from the wells of the fusion plates and replaced with fresh medium. On day 9, the fusion was screened by ELISA, testing for the presence of mouse IgG preferentially binding to the C11/MBP fusion protein compared to maltose binding protein alone. Immulon 4 plates (Dynatech, Cambridge, Mass.) were coated overnight at 4° C. with 100 ng/well C11/MBP or maltose binding protein diluted in 50 mM carbonate buffer, pH 9.6. Plates were washed three times with PES with 0.05% Tween 20 (PBST) and 50 μl culture supernatant was added. After incubation at 37° C. for 30 minutes, and washing as above, 50 μl of horseradish peroxidase conjugated goat anti-mouse IgG(fc) (Jackson ImmunoResearch, West Grove, Pa.) diluted 1:3500 in PBST was added. Plates were incubated as above, washed four times with PBST and 100 μl substrate [consisting of 1 mg/ml o-phenylene diamine (Sigma) and 0.1 μl/ml 30% H₂O₂ in 100 mM Citrate, pH 4.5] was added. The color reaction was stopped after five minutes with the addition of 50 μl of 15% H₂SO₄. A₄₉₀ was read on a plate reader (Dynatech). On day 9 the fusion was also screened by ELISA on IMR90 cell (ATCC, Manassas, Va.) monolayers in 96 well plates. Cell monolayers were fixed with 4% paraformaldehyde and permeabilized for 15 min. on ice with CKS buffer: 10 mM Pipes, pH 6.8, 300 mM sucrose, 100 mM NaCl, 3 mM MgCl₂, 0.5% Triton X-100. Hybridoma supernatants were tested using the above protocol for ELISA but with the following changes: incubation times were one hour at room temperature, all washes were done three times, and the amount of goat anti-mouse and substrate were 150 μl each. Fusion wells were selected that were positive on C11/MBP and IMR90 cells, but negative on maltose binding protein. Selected fusion wells were cloned twice by dilution into 96 well plates and visually scoring the number of colonies/well after 5 days. Clonal cell lines resulted from seven original fusion wells. These lines were designated 113B, 113E, 113F, 113H, 113I, 113J, and 113L. Two of these hybridoma cell lines, C11-113E and C11-113H, were deposited with the American Type Culture Collection (ATCC), 10801 University Boulevard, Manassas, Va., 20110-2209, U.S.A. on Apr. 21, 1998 and were assigned deposit numbers of HB-12515 and HB-12514, respectively.

The monoclonal antibodies produced by hybridomas were isotyped in an ELISA assay. Immulon 4 plates were coated at 4° C. with 50 μl/well goat anti-mouse IgA,G,M (Organon Teknika) diluted 1:5000 in 5 mM carbonate buffer, pH 9.6. The assay was performed as described above. Monoclonal antibodies were detected with horseradish peroxidase conjugated rabbit anti-mouse IgG₁, G₂ₐ, or G₃ (Zymed, San Francisco, Calif.) diluted 1:1000 in PBST with 1% normal goat serum. Results showed that the monoclonal antibodies produced by hybridomas from fusion 113 and were all IgG₁.

### EXAMPLE 11

A. Cad-11 Expression Vector (pCAD-11)

The full length cad-11 cDNA (SEQ ID NO:1), was subcloned into the eukaryotic expression vector, pSPORT (Gibco/BRL Burling,-on, Ont.) using EcoR1 cloning sites. The orientation of the cad-11 insert was confirmed by DNA sequence analysis. A clone which contained the cad-11 insert in the sense direction was identified (pCAD-11) A vector containing the LacZ gene, pLacZ was prepared in the same way and was used to determine the transfection efficiency by co-transfection with the cad-11 vector.

COS-11 cells were transfected with the two expression vectors, pCAD-11, and pLacZ following the methods described by MacCalman et al. (1996) “Differentiation-Dependent Transuction of Human Trophoblast Cells by Recombinant Adenovirus”. Biology of Reproduction 54:682-69. The cells were washed twice with serum-free DMEM. The cells were then incubated in 1 ml of serum free-medium containing 2 μg of plasmid and 10 μg Lipofectamine™ (GIBCO/BRL). After 5 h incubation, 1 ml of culture medium containing 20% FCS was added to the cells. Twenty four h after transfection, the cells were harvested for Western blot analysis and immunohistochemisty performed as described in the preceding example

Western blot analysis revealed a single cad-11 protein species of 125 kDa in extracts prepared from COS cells transfected with pCAD-11 but not in cells transfected with pLacZ. Immunohistochemistry demonstrated that cad-11 was expressed on the cell surface of cells transfected with pCAD-11. Thus, pCAD-11 is capable of directing the production of the mature cad-11 protein species in transfected cells.

B. Recombinant Adenovirus Vector: (Ad.CMVlac Z)

The production of a replication-deficient adenovirus vector containing the E. coli LacZ cDNA has been previously described (MacCalman et al. (1996) [supra]). The vector is constructed from an adenovirus type 5 (Ad5) mutant, which lacks most of the viral sequence regions E1a and E1b and a portion of E3. By homologous recombinant techniques, the E. coli LacZ cDNA, driven by the human CMV3′ promoter region was inserted into the viral genome. Cad-11 DNA driven by the same or similar promoter may be similarly inserted into the virus.

(1) Large Scale Production, Purification and Titration of Ad.CMVlacZ: Human embryonic kidney 293 cells were grown to 90% confluency in 150 mm culture dishes containing DMEM supplemented with 10% FCS. Immediately before infection with the recombinant Ad vectors (1×10¹⁰ viral particle/plate), the culture medium was replaced with DMEM containing 2% FCS. Thirty six hours after infection, immediately before the cytophathic effect was complete, the cells were scraped and pelleted by centrifugation at 4,000×g for 20 min at 4° C. The cell pellet was freeze-thawed three times and subjected to centrifugation at 3,000×g for 10 min at 4° C. The supernatant, which contained the virus, was layered onto a discontinuous CsCl gradient and subjected to ultracentrifugation at 50,000×g for 4 h at 4° C. The collected viral band was subjected once more to the same gradient centrifugation for 15 h at 4° C. THe collected viral band was desalted in a Sephadex G25 column. The viral concentration was determined by spectrophotemetry (at 260 nm).

(2) Gene Transfer into Endometrial Stromal Cells: Ad-mediated gene transfer into endometrial stromal cells was evaluated by detection of vector-specific protein expression. To accomplish this, endometrial stromal cells were cultured in 2.5 cm² plastic dishes (Falcon, Bectin Dickinson, Lincoln Park, N.J.). At 50-60% confluency (approximately 2×10⁶ cells), the cells were infected with 1×10⁴, 2×10⁴ or 4×10⁴ viral particles/cell of Ad.CMVlacZ. To detect expression of β-gal, 48 h after exposure to the recombinant Ad vectors, the cells were fixed and stained with the β-gal substrate, X-gal (5-bromo-4-chloro-3indolyl-β-D-galactosidase). The presence of β-gal activity is indicted by a blue stain that is present in cells in which gene transfer and expression has been successful.

There was a dose-response relationship between the number of viral particles/cell and the transduction efficiency of endometrial stromal cells. The number of stained cells, as well as the intensity of staining, was greatest in cells infected with 4×10⁴ viral particles/cells. A cytopathic effect was not observed at any of the virus concentration used in this study. X-gal staining was not observed in untreated cells, demonstrating that endogenous β-gal activity makes no significant contribution to the X-gal staining.

All publications and patents cited in this specification are incorporated herein by reference. Although the foregoing invention has been described in some detail by way of illustration and example for purposes of clarity of understanding, it will be readily apparent to those of ordinary skill in the art in light of the teachings of this invention that changes and modification may be made thereto without departing from the spirit or scope of the appended claims.

What is claimed is:

1. A method of determining likelihood of establishment or maintenance of a pregnancy comprising determining the level of cadherin-11 mRNA or protein from endometrial cells of a female subject and comparing said level from the female subject to a standard level indicative of ability to establish or maintain a pregnancy in a female, wherein a reduced level relative to said standard level of cadherin-11 in the female subject indicates inability to establish or maintain a pregnancy.

2. The method of claim 1, wherein the inability to establish or maintain a pregnancy in the female subject is modifiable by a progestin.

3. The method of claim 1, wherein the inability to establish or maintain a pregnancy in the female subject is due to luteal phase deficiency.

4. The method of claim 1, wherein level of cadherin-11 protein of the female subject is determined using an antibody that binds to cadherin-11.

5. The method of claim 4 wherein the antibody is selected from the group consisting of a monoclonal antibody produced by a hybridoma deposited under ATCC HB-12514 and a monoclonal antibody produced by a hybridoma deposited under ATCC HB-12515.

6. The method of claim 1, wherein level of cadherin-11 mRNA of the female subject is determined using a polynucleotide that hybridizes to cadherin-11 mRNA.

7. The method of claim 6, wherein said polynucleotide comprises the sequence set forth in SEQ ID NO: 1, or a fragment thereof.

8. The method of claim 1, wherein level of cadherin-11 protein of the female subject is determined by determining cadherin-11 protein level in a blood sample from said female subject.

9. The method of claim 8, wherein the cadherin-11 protein level from the female subject is compared to a standard level associated with a blood sample from a fertile female subject.

10. A method of diagnosing a reason for inability to establish or maintain a pregnancy in a female subject comprising detecting in cells from the subject, a defect selected from the group consisting of the absence of a gene encoding cadherin-11 and the presence of a mutation in said gene.

11. The method of claim 10 wherein DNA from said female subject is annealed to a polynucleotide selected from the group consisting of the DNA of SEQ ID NO: 1, a fragment of said DNA, and polynucleotides complementary thereto.

12. A method for determining likelihood of establishing or maintaining pregnancy, or endometrial receptivity to blastocyst implantation in a female subject receiving fertility increasing therapy, comprising determining the level of cadherin-11 mRNA or protein in endometrial cells from the female subject.

13. The method of claim 12 further comprising comparing said level from the female subject to a standard level indicative of a fertile female.

14. The method of claim 12, wherein said fertility increasing therapy is to increase progestin levels in the female subject.

15. The method of claim 13, further comprising the step of adjusting the fertility increasing therapy to increase the level of cadherin-11 produced by endometrial cells in the female subject.

16. The method of claim 13, further comprising the step of determining the optimal time for blastocyst implantation.

17. A method of delivering DNA encoding cadherin-11 to endometrial cells comprising contacting said cells with a DNA construct comprising said DNA.

18. The method of claim 17, wherein the endometrial cells are in a female subject.

19. The use of DNA encoding cadherin-11 to prepare a medicament for use in the method of claim 18.

20. The use of a progestin to increase cad-11 production in endometrial tissue.

21. The use of a progestin to prepare a medicament to increase cad-11 production in endometrial tissue.

22. A kit for performing a determination of an inability of a female to establish or maintain a pregnancy comprising an agent for measuring cadherin-11 mRNA or protein selected from the group consisting of:
(i) an antibody that binds to cadherin-11; and
(ii) a polynucleotide that hybridizes to cadherin-11 mRNA; and,
wherein the kit further comprises a standard sample containing levels of cadherin-11 mRNA or protein indicative of ability of a female to establish or maintain a pregnancy.


SEQUENCE LISTING (4 sequences)

SEQ ID NO: 1 - DNA, 2625 bp, Homo sapiens
cggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagaggctataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccgaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagcccaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaacaggtatttttt

SEQ ID NO: 2 - DNA, 23 bp, Artificial Sequence
  Description of Artificial Sequence primer
ctcctccgtattactccattcaa

SEQ ID NO: 3 - DNA, 22 bp, Artificial Sequence
  Description of Artificial Sequence primer
atttgctccaggtgtcaagaca

SEQ ID NO: 4 - protein, 796 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQRLYQMSVSEAAVPGEEVGRVKAKDPDIGENGLVTYNIVDGDGMESFEITTDYETQEGVIKLKKPVDFETERAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAQVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS